(19) United States
(12) Patent Application Publication
(10) Pub. No.: US 2023/0351305 A1
(43) Pub. Date: Nov. 2, 2023

(54) DATA ANALYSIS AND REPORTING SYSTEMS AND METHODS

(71) Applicant: R.P. Scherer Technologies, LLC, Carson City, NV (US)

(72) Inventors: Amin ABEDINI, Lexington, KY (US); Christin HOLLIS, Paris, KY (US); Matthew DEFRESE, Lexington, KY (US)

(73) Assignee: R.P. Scherer Technologies, LLC, Carson City, NV (US)

(21) Appl. No.: 18/140,780

(22) Filed: Apr. 28, 2023

Related U.S. Application Data
(60) Provisional application No. 63/336,035, filed on Apr. 28, 2022.

(51) Int. Cl. G06Q 10/0639; G06Q 50/04; G16H 15/00

(52) U.S. Cl. CPC G06Q 10/06395; G06Q 50/04; G16H 15/00

(57) ABSTRACT

Systems and methods for automated data analysis and reporting of pharmaceutical batch production records are provided. In response to receiving a user command to generate a batch report, the method can comprise automatically performing one or more statistical analyses on raw batch data extracted from one or more batch manufacturing records, generating one or more figures and one or more tables using the analyzed data, identifying one or more discussion boxes that are relevant to the analyzed data, compiling a batch report comprising the one or more identified discussion boxes, saving the batch report in a memory, and displaying the batch report on a user device.

### CROSS-REFERENCE TO RELATED APPLICATIONS

This application claims the benefit of U.S. Provisional Application No. 63/336,035, filed Apr. 28, 2022, the entire contents of which is incorporated herein by reference.

### FIELD OF THE DISCLOSURE

This disclosure relates to systems and methods for automated data analysis and reporting of pharmaceutical batch production records.

### BACKGROUND OF THE DISCLOSURE

When manufacturing pharmaceutical drug compositions, part of drug development is performing a feasibility study of the manufacturing process. Performing a feasibility study involves reviewing and analyzing a batch production record, which provides a complete history of each stage of the manufacturing process of a given pharmaceutical lot, or batch, that is produced. Batch production records are used to ensure health, safety, and quality, and provide an auditable record detailing compliance with federal regulations. The results of a given feasibility study will identify a manufactured drug's compliance or deviation from the specifications for that drug. A feasibility study can thus be used to attest to compliance with the specifications for a given drug, or to assess discrepancies from those specifications.

### SUMMARY OF THE DISCLOSURE

The process of creating a feasibility report generally includes batch testing, batch data collection, data analysis, and report drafting. A given feasibility report can contain analyzed batch data as well as discussion of the results, including discussion regarding compliance with specifications, and explanations of the manufacturing and analysis processes.

Currently, each of the batch data collection, data analysis, and report drafting steps of the report drafting process are performed manually. Batch data is collected from batch production records, which generally take the form of a printed document or a portable document format (PDF) document. Such document format is necessary to ensure that batch production records meet regulatory requirements and can be audited regularly. Collecting batch record data from a printed or PDF document requires manually reading the document, identifying the relevant data, and then copying that data into a computing system so that it can be analyzed. This process is generally performed by two individuals, with one individual manually copying data values into a spreadsheet and another individual reviewing both the batch production record and the spreadsheet values to ensure the individuals copied the values accurately.

After copying batch record data into a spreadsheet, one or more individuals may then perform data analysis. Such data analysis can include, for example, assessing physical properties such as tablet weight, hardness, and/or thickness, as well as assessing blend uniformity, stratified content uniformity, dissolution, water content, and/or disintegration time. The sophistication level of the data analysis is individual-driven. That is, highly skilled data scientists will provide analysis that is more sophisticated than less skilled data scientists will. As analysis that is more sophisticated leads to higher quality reports, more highly trained data scientists are necessary to ensure high quality reports are produced. This leads to increased training costs and increased demand for more highly trained individuals. Furthermore, because manual data analysis is individual-driven, there exists a lack of consistency between the data analysis performed by different individuals.

Variability owing to the individual-driven nature of manual data analysis carries over into the report drafting stage of the feasibility report process. Thus, the individual-driven nature of the feasibility report process can introduce a lack of consistency in both the data analysis contained in the report and any discussion of that analysis in the report. Such inconsistencies can compromise both the scientific quality of feasibility reports and the business reputation of the entity creating the reports.

Accordingly, systems and methods for automated data analysis and reporting of pharmaceutical batch production records are presented herein. By automating the report production process, companies can realize significant time savings and improve the quality and consistency of the reports and ensure the reports meet scientific standards for analysis and formatting. Additionally, companies can improve client satisfaction by shortening the turnaround time to generate reports and delivering higher quality reports. Companies can also minimize the need for highly skilled individuals, decrease training costs, increase productivity, and boost their business reputation.

In one or more examples, a computer-implemented method for generating a batch report comprises: extracting raw batch data from one or more batch manufacturing reports received from one or more manufacturing devices and storing the extracted raw batch data in a memory, receiving a user command on a user device to automatically generate a batch report, and in response to receiving the user command: performing one or more statistical analyses on the stored raw batch data, generating one or more figures and one or more tables using the analyzed data and storing the one or more figures and the one or more tables in the memory, identifying one or more discussion boxes that are relevant to the analyzed data, compiling a batch report comprising the one or more identified relevant discussion boxes, the one or more figures, and the one or more tables, saving the compiled batch report in the memory, and displaying the compiled batch report on the user device.

Optionally, the computer-implemented method comprises identifying one or more outlier values in the analyzed data, and determining whether the one or more outlier values are caused by a manufacturing process error or a post-manufacturing data analysis error.

In one or more examples, the raw batch data can be extracted from the one or more batch manufacturing reports in real-time as the one or more batch manufacturing reports are received and the computer-implemented method can comprise: providing one or more guidelines to adjust one or more settings of the one or more manufacturing devices if the outlier value is caused by a manufacturing process error.

The one or more batch manufacturing reports can comprise a plurality of batch pages, and extracting the raw batch data comprises: receiving one or more image coordinates corresponding to one or more bounded areas within a page, photoscanning each page of the plurality of batch pages, collecting raw batch data located within the one or more bounded areas on each page of the plurality of photoscanned batch pages, and generating a ledger containing the collected raw batch data.

Optionally, compiling the batch report comprises generating one or more instruction codes that comprise instructions to: populate one or more modifiable handles in the one or more relevant discussion boxes with one or more statistical data values from the analyzed data, embed the one or more figures and the one or more tables in one or more appropriate locations based on the relevant discussion boxes and generate a data report comprising the one or more populated relevant discussion boxes, the one or more embedded figures, and the one or more embedded tables.

In one or more examples, the one or more instruction codes comprise LaTeX codes.

Optionally, the one or more modifiable handles can each correspond to a data metric, the one or more discussion boxes comprise pre-written text regarding one or more data metrics, and populating the one or more modifiable handles comprises replacing each of the modifiable handles with a corresponding data metric from the analyzed data.

In one or more examples, the compiled batch report can indicate whether the analyzed data is within one or more defined batch specifications.

In one or more examples, a system for generating a batch report, the system comprises: a memory, one or more processors, and one or more programs, wherein the one or more programs are stored in the memory and configured to be executed by the one or more processors, and the one or more programs when executed by the one or more processors cause the processor to: extract raw batch data from one or more batch manufacturing reports received from one or more manufacturing devices and storing the extracted raw batch data in a memory, receive a user command on a user device to automatically generate a batch report, and in response to receiving the user command: perform one or more statistical analyses on the stored raw batch data, generate one or more figures and one or more tables using the analyzed data and storing the one or more figures and the one or more tables in the memory, identify one or more discussion boxes that are relevant to the analyzed data, compile a batch report comprising the one or more relevant discussion boxes, the one or more figures, and the one or more tables, save the compiled batch report in the memory, and display the compiled batch report on the user device.

Optionally, the one or more programs when executed by the one or more processors cause the processor to: identify one or more outlier values in the analyzed data, and determine whether the one or more outlier values is caused by a manufacturing error or a post-manufacturing data analysis error.

In one or more examples, the raw batch data can be extracted from the one or more batch manufacturing reports in real-time as the one or more batch manufacturing reports are received.

The one or more programs can, when executed by the one or more processors, cause the processor to provide one or more guidelines to adjust one or more settings of the one or more manufacturing devices if the outlier value is caused by a manufacturing process error.

Optionally, the one or more batch manufacturing reports can comprise a plurality of batch pages, and extracting the raw batch data can comprise: receiving one or more image coordinates corresponding to one or more bounded areas within a page, photoscanning each page of the plurality of batch pages, collecting raw batch data located within the one or more bounded areas on each page of the plurality of photoscanned batch pages, and generating a ledger containing the collected raw batch data.

In one or more examples, compiling the batch report comprises generating one or more instruction codes that comprise instructions to: populate one or more modifiable handles in the one or more relevant discussion boxes with one or more statistical data values from the analyzed data, embed the one or more figures and the one or more tables in one or more appropriate locations based on the relevant discussion boxes, and generate a data report comprising the one or more populated relevant discussion boxes, the one or more embedded figures, and the one or more embedded tables.

Optionally, the one or more instruction codes can comprise LaTeX codes.

In one or more examples, the one or more modifiable handles can each correspond to a data metric, the one or more discussion boxes can comprise pre-written text regarding one or more data metrics, and populating the one or more modifiable handles can comprise replacing each of the modifiable handles with a corresponding data metric from the analyzed data.

In one or more examples, the compiled batch report can indicate whether the analyzed data is within one or more defined batch specifications.

In one or more examples, a computer-readable storage medium storing one or more programs for generating a batch report, the one or more programs comprising instructions which, when executed by an electronic device with a display and a user input interface, cause the device to: extract raw batch data from one or more batch manufacturing reports received from one or more manufacturing devices and storing the extracted raw batch data in a memory, receive a user command on a user device to automatically generate a batch report, and in response to receiving the user command: perform one or more statistical analyses on the stored raw batch data, generate one or more figures and one or more tables using the analyzed data and storing the one or more figures and the one or more tables in the memory, identify one or more discussion boxes that are relevant to the analyzed data, compile a batch report comprising the one or more relevant discussion boxes, the one or more figures, and the one or more tables, save the compiled batch report in the memory, and display the compiled batch report on the user device.

Optionally, the one or more programs when executed by the electronic device cause the device to: identify one or more outlier values in the analyzed data, and determine whether the one or more outlier values is caused by a manufacturing error or a post-manufacturing data analysis error.

In one or more examples, the raw batch data can be extracted from the one or more batch manufacturing reports in real-time as the one or more batch manufacturing reports are received.

In one or more examples, the one or more programs when executed by the electronic device can cause the device to provide one or more guidelines to adjust one or more settings of the one or more manufacturing devices if the outlier value is caused by a manufacturing process error.

The one or more batch manufacturing reports can comprise a plurality of batch pages, and extracting the raw batch data can comprise: receiving one or more image coordinates corresponding to one or more bounded areas within a page, photoscanning each page of the plurality of batch pages, collecting raw batch data located within the one or more bounded areas on each page of the plurality of photoscanned batch pages, and generating a ledger containing the collected raw batch data.

In one or more examples, compiling the batch report can comprise generating one or more instruction codes that comprise instructions to: populate one or more modifiable handles in the one or more relevant discussion boxes with one or more statistical data values from the analyzed data, embed the one or more figures and the one or more tables in one or more appropriate locations based on the relevant discussion boxes, and generate a data report comprising the one or more populated relevant discussion boxes, the one or more embedded figures, and the one or more embedded tables.

Optionally, the one or more instruction codes can comprise LaTeX codes.

In one or more examples, the one or more modifiable handles can each correspond to a data metric, the one or more discussion boxes can comprise pre-written text regarding one or more data metrics, and populating the one or more modifiable handles can comprise replacing each of the modifiable handles with a corresponding data metric from the analyzed data.

In one or more examples, the compiled batch report can indicate whether the analyzed data is within one or more defined batch specifications.

It will be appreciated that any of the variations, aspects, features and options described in view of the systems can be combined.

Additional advantages will be readily apparent to those skilled in the art from the following detailed description. The aspects and descriptions herein are to be regarded as illustrative in nature and not restrictive.

All publications, including patent documents, scientific articles and databases, referred to in this application are incorporated by reference in their entirety for all purposes to the same extent as if each individual publication were individually incorporated by reference. If a definition set forth herein is contrary to or otherwise inconsistent with a definition set forth in the patents, applications, published applications and other publications that are herein incorporated by reference, the definition set forth herein prevails over the definition that is incorporated herein by reference.

### BRIEF DESCRIPTION OF THE FIGURES

The invention will now be described, by way of example only, with reference to the accompanying drawings, in which:

FIG. 1 depicts an exemplary automated process to generate a batch report, in accordance with one or more examples of the disclosure.

FIG. 2 depicts exemplary user interfaces for a user to command the automated generation of a batch report, in accordance with one or more examples of the disclosure.

FIG. 3 depicts an exemplary user interface for a user to input data required for an automated process to generate a batch report, in accordance with one or more examples of the disclosure.

FIG. 4 depicts an exemplary user interface comprising figures generated using an automated data analysis method, in accordance with one or more examples of the disclosure.

FIG. 5 depicts an exemplary user interface comprising an exemplary table of contents page generated using an automated data analysis method, in accordance with some examples of the disclosure.

FIG. 6 depicts an exemplary user interface comprising exemplary page from a manufacturing steps and results section of a report generated using an automated data analysis method, in accordance with some examples of the disclosure.

FIG. 7 depicts an exemplary user interface comprising an exemplary page from an analytical results section of a report generated using an automated data analysis method, in accordance with one or more examples of the disclosure.

FIG. 8 depicts an exemplary automated process to extract raw batch data from a printed or PDF batch production report, in accordance with one or more examples of the disclosure.

FIG. 9 depicts an exemplary user interface comprising a batch production report, in accordance with one or more examples of the disclosure.

FIG. 10 depicts an exemplary process for a user to command the automated generation of a batch report, in accordance with one or more examples of the disclosure.

FIG. 11 illustrates an example of a computing device, in accordance with one or more examples of the disclosure.

### DETAILED DESCRIPTION OF THE DISCLOSURE

As explained above, current methods to generate a feasibility report require manual data collection, analysis, and report drafting. The individual-driven nature of these methods can result in sub-standard data analysis, sub-standard graphical and/or typesetting quality, compromised scientific quality, and long turnaround periods for the generation of a given report.

The previous manual process to generate a feasibility report required an individual to: (1) manually collect data values from a batch production report, (2) verify the manually collected data values, (3) perform data analysis, (4) create figures and tables, and (5) draft the feasibility report. Whereas manually generating a report previously required the user to follow a five-step process, automating report generation can simplify the process down to just two steps by an individual: (1) executing a program, and (2) reviewing the generated batch data report. Such automation can drastically reduce the time any given user has to spend generating a report. For example, manually drafting a given report may take weeks from start to finish, but automatically generating the same report could take only hours to days.

An automated report generator can perform each step required to generate a high quality report without requiring significant input from a user. For example, an automated report generator can accurately extract data values from batch production records, automatically perform complicated data analysis, automatically generate high quality figures and tables based on that analysis, incorporate relevant discussion of the analysis, and compile a report that includes the data analysis and discussion and the figures and tables that were automatically generated. Thus, an automated report generator can reduce the human interaction required down to simply executing the program and reviewing the report.

In addition to reducing the time and effort required to draft a report, the automated report generator can also improve the quality and consistency of the reports generated. For example, as explained above, highly skilled data scientists will provide more sophisticated analysis than less skilled or newer data scientists will. Moreover, because the data analysis and written discussion performed by individuals can vary, manually drafted reports lack consistency when drafted by multiple individuals. This problem is entirely removed via an automated report generator, however, because the automated report generator can repeat the same, sophisticated, analysis each time it is executed, and discuss the results in the same manner, resulting with consistent high quality reports.

By automating the report production process, companies can thus realize significant time savings, improve the quality and consistency of the reports, and ensure the reports meet scientific standards for analysis and formatting. Additionally, companies can improve client satisfaction by shortening the turnaround time to generate reports, and delivering higher quality reports. Companies can also minimize the need for highly skilled individuals, decrease training costs, increase productivity, and boost their business reputation.

In the following description of the disclosure and examples, reference is made to the accompanying drawings in which are shown, by way of illustration, specific examples that can be practiced. It is to be understood that other examples can be practiced, and changes can be made, without departing from the scope of the disclosure.

In addition, it is also to be understood that the singular forms “a,” “an,” and “the,” used in the following description are intended to include the plural forms as well unless the context clearly indicates otherwise. It is also to be understood that the term “and/or” as used herein refers to and encompasses any and all possible combinations of one or more of the associated listed items. It is further to be understood that the terms “includes,” “including,” “comprises,” and/or “comprising,” when used herein, specify the presence of stated features, integers, steps, operations, elements, components, and/or units but do not preclude the presence or addition of one or more other features, integers, steps, operations, elements, components, units, and/or groups thereof.

Some portion of the detailed description that follows are presented in terms of algorithms and symbolic representations of operations executed on data bits within a computer memory. These algorithmic descriptions and representations are the means used by those skilled in the data processing arts to most effectively convey the substance of their work to others skilled in the art. An algorithm is here, and generally, conceived to be a self-consistent sequence of steps (instructions) leading to a desired result. The steps are those requiring physical manipulations of physical quantities. Usually, though not necessarily, these quantities take the form of electrical, magnetic, or optical signals capable of being stored, transferred, combined, compared, and otherwise manipulated. It is convenient at times, principally for reasons of common usage, to refer to these signals as bits, values, elements, symbols, characters, terms, numbers, or the like. Furthermore, it is also convenient at times to refer to certain arrangements of steps requiring physical manipulations of physical quantities as modules or code devices without loss of generality.

However, all of these similar terms are to be associated with the appropriate physical quantities and are merely convenient labels applied to these quantities. Unless specifically otherwise apparent from the following discussion, it is to be appreciated that throughout the description, discussions utilizing terms such as “processing,” “computing,” “calculating,” “determining,” “displaying,” or the like, refer to the action and processes of a computer system or similar electronic computing device that manipulates and transforms data represented as physical (electronic) quantities within the computer system memories or registers, or other information storage, transmission, or display devices.

Certain aspects of the present disclosure include process steps and instructions described in the form of an algorithm. It should be noted that the process steps and instructions of the present disclosure could be embodied in software, firmware, or hardware, and, when embodied in software, they could be downloaded to reside on and be operated from different platforms used by a variety of operating systems.

The present disclosure also relates to a device for performing the operations herein. The device may be specially constructed for the required purposes or it may comprise a general-purpose computer selectively activated or reconfigured by a computer program stored in the computer. Such a computer program may be stored in a non-transitory, computer-readable storage medium such as, but not limited to, any type of disk including floppy disks, optical disks, CD-ROMs, magnetic-optical disks, read-only memories (ROMs), random access memories (RAMs), EPROMs, EEPROMs, magnetic of optical cards, application-specific integrated circuits (ASICs), or any type of media suitable for storing electronic instructions and each coupled to a computer system bus. Furthermore, the computers referred to in the specification may include a single processor or may be architectures employing multiple processor designs for increased computing capability.

The methods, devices, and systems described herein are not inherently related to any particular computer or other apparatus. Various general-purpose systems may also be used with programs in accordance with the teachings herein, or it may prove convenient to construct a more specialized apparatus to perform the required method steps. The required structure for a variety of these systems will appear from the description below. In addition, the present disclosure is not described with reference to any particular programming language. It will be appreciated that a variety of programming languages may be used to implement the teachings of the present disclosure as described herein.

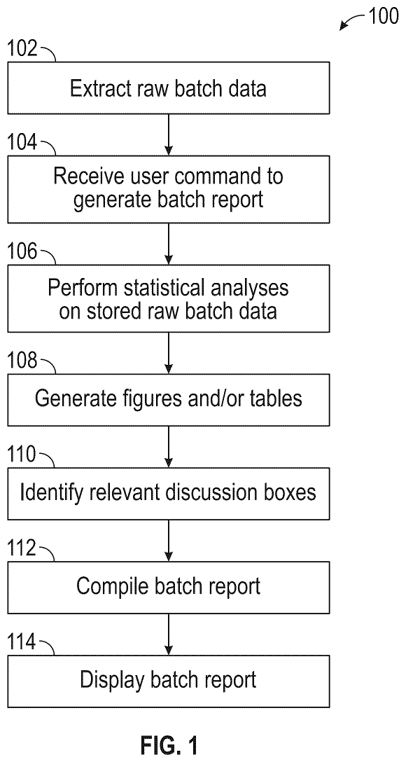

FIG. 1 depicts an exemplary automated process 100 to generate a batch report, in accordance with one or more examples. Automated process 100 can be performed by any suitable type of microprocessor-based device, such as a personal computer, workstation, server, or handheld computing device (portable electronic device) such as a phone or tablet.

As shown in FIG. 1, the automated process 100 begins at step 102 with extracting raw batch data. Raw batch data can be extracted from a batch production report received from a variety of manufacturing devices. Example devices can include, in non-limiting examples, a tablet tester, a fluid bed processor, a vertical granulator, a v-blender/bin-blender, a capsule weight sorter, a roller compactor, an extruder, a pan coating process tester, and/or a high performance liquid chromatography (HPLC) device. The raw data extracted can include, in non-limiting examples, measurements of tablet weight, thickness, and/or hardness, measurements of air flow, inlet air temperature, product temperature, exhaust temperature, and/or dew point, in-process measurements of blade speed, chopper speed, water addition rate, and/or blade/chopper torque. In one or more examples, the raw data extracted at step 102 can be saved in a memory in a location and/or format that can be accessed by scientists and/or users. In one or more examples, extracting raw batch data at step 102 can occur in response to a user command to extract the batch data. Extracting the raw batch data at step 102 can also occur automatically upon receiving a batch production report.

After extracting raw batch data at step 102, the automated process 100 can move to step 104 and receive a user command to generate a batch report. The user command received at step 104 of automated process 100 can be received via a command from a user interacting with a user interface of any suitable computing device. The computing device that receives the user command can be the same computing device that is performing the automated process 100, or can be communicatively coupled to the computing device that is performing the automated process 100. In one or more examples, extracting the raw batch data at step 102 can occur in response to receiving a user command to generate a batch report.

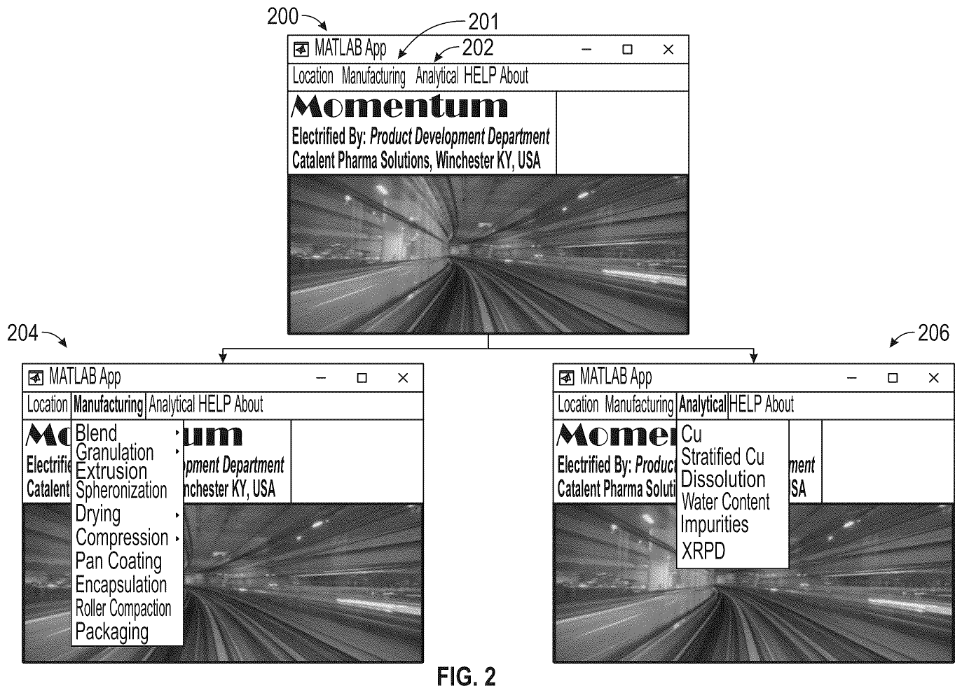

FIG. 2 depicts exemplary user interfaces for a user to command the automated generation of a batch report, in accordance with one or more examples. The user interfaces shown in FIG. 2 can be used by a user to generate the user command that is received at step 104 of the automated process 100. As shown in FIG. 2, the user interface 202 enables a user to select an analysis type of manufacturing 201 or analytical 202. Manufacturing user interface 204 and analytical user interface 206 then enable a user to select a specific analysis based on the selected analysis. For example, if a user selects manufacturing 201, the user can then be presented with the manufacturing user interface 204 with a list of possible specific manufacturing analyses, such as analyzing blend, granulation, extrusion, spheronization, drying, compression, pan coating, encapsulation, roller compaction, packaging, etc. If a user selects analytical 202, the user may be presented with the analytical user interface 206 with a list of possible specific analytical analyses, such as analyzing content uniformity, stratified content uniformity, dissolution, water content, impurities, an x-ray powder diffraction (XRPD) analysis, etc.

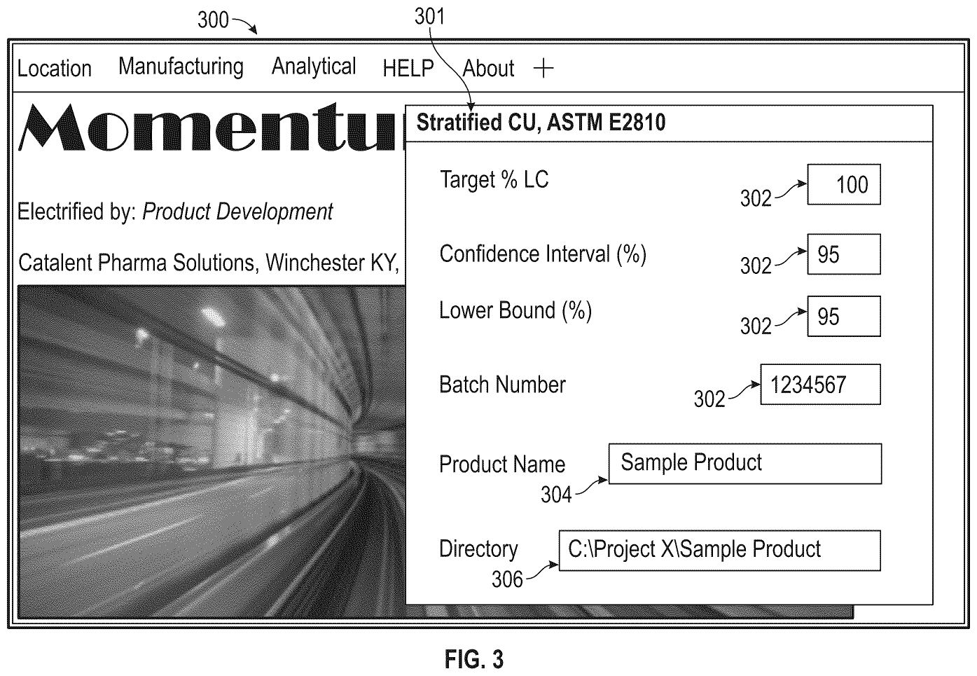

In one or more examples, upon selecting an analysis type, a user may be prompted to provide some project information relating to the batch report that the user commands to be generated at step 104. FIG. 3 depicts an exemplary user interface 300 for a user to input data required for an automated process to generate a batch report, in accordance with one or more examples. The user interface 300 may be displayed after a user selects an analysis type and a specific analysis as discussed above. As shown in in FIG. 3, the user interface 300 includes a window 301 that includes data fields 302 for a user to add required data, a product name field 304, and a directory field 306. The required data the user can add in data fields 302 can vary based on the specific analysis type. The directory field 306 can enable the user to dictate where the files generated by the automated process will be saved. Such files can include, for example, spreadsheets, figures, a document, etc. After entering information in the window 301, the user may select a button or region of the user interface 300 to run an automated process. Upon selecting a button to run an automated process, a command can be received at step 104 of the automated process 100.

Referring back to FIG. 1, in response to the user command received at step 104, the automated process 100 can move to step 106 and perform statistical analyses on the stored raw batch data that was extracted at step 102. The statistical analyses can be pre-determined by one or more scientists in advance of being implemented by the automated process 100. The statistical analyses can include, for example, assessing variations in physical properties such as tablet weight, hardness, and/or thickness over time. In one or more examples, the statistical analyses can include assessing blend uniformity, stratified content uniformity, dissolution, water content, and/or disintegration time. In one or more examples, data corresponding to the statistical analyses performed at step 106 can be saved in a memory in a location and/or format that can be accessed by scientists and/or users to enable those scientists and/or users to review the data used to generate the final batch report. The scientists and/or users thus may make any necessary or desirable edits to the analyses, which can be used to generate a new version of the batch report.

After performing statistical analyses on the stored raw batch data at step 106, the automated process 100 can move to step 108 and generate one or more figures and/or tables. In one or more examples, generating figures and/or tables at step 108 can be included as part of performing statistical analyses at step 106. The one or more tables created by the automated process 100 can, in one or more examples, tabulate data used in the figures, various parameters, analytical summaries, etc. The one or more figures can include, in non-limiting examples, visualization of particle size analysis, various in-process parameters over time, and/or physical property statistics.

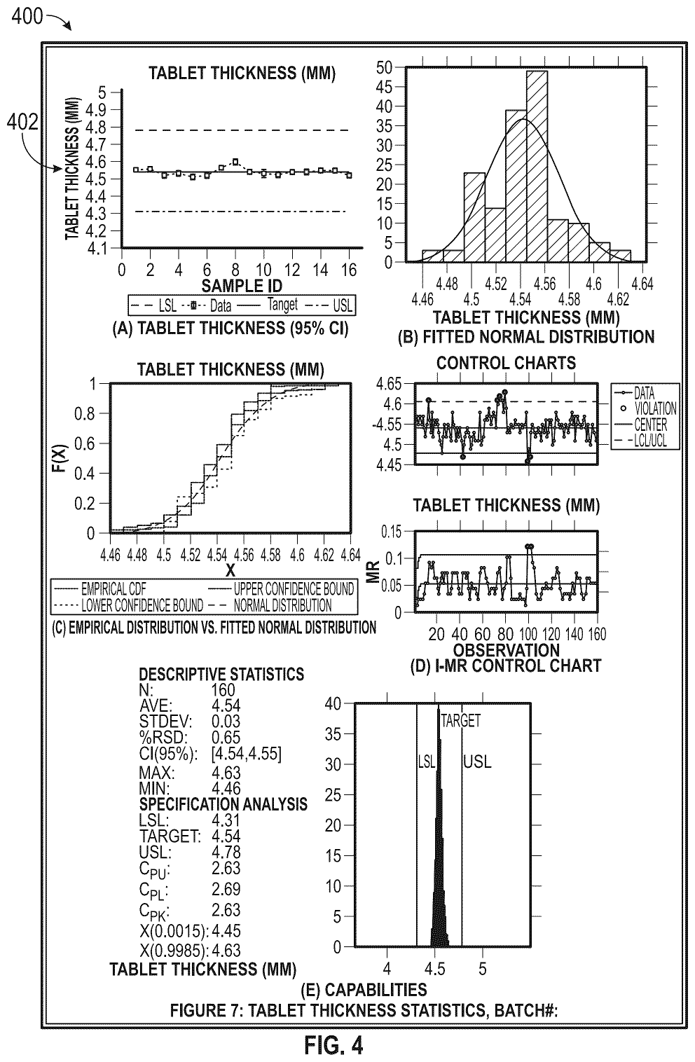

FIG. 4 depicts an exemplary user interface comprising a figures page 400 generated using an automated data analysis method, in accordance with one or more examples. Figures page 400 can be generated via automated process 100. As shown in FIG. 4, the figures page 400 provides visualizations regarding the tablet thickness characteristics of a given batch. For example, the figures page 400 includes a graph 402 of tablet weight over time, which graphically depicts tablet thickness values over time compared to the target tablet thickness specifications, including upper and lower specification limits. In one or more examples, the figures and/or tables generated at step 108 can be saved in a memory in a location and/or format that can be accessed by scientists and/or users. The scientists and/or users can thus review the tables and/or figures separately from the final report, and may make any necessary or desirable edits to the tables and/or figures, which can be used to generate a new version of the batch report.

As explained above, previously the level of sophistication of the data analysis performed via the manual process was individual-driven, with sophistication level correlated with the skill level of a given individual. In the automated process 100, however, the data analysis can be designed by one or more highly skilled individuals with the automated process 100 configured to repeat the same analysis each time it generates a report. Accordingly, the automated process 100 can ensure that the sophistication level of the analyses is high. Moreover, by standardizing the analysis methods and scope and repeating the same analyses in an automated fashion, the automated process 100 removes the inconsistencies inherent in the prior individual-driven manual process. By removing these inconsistencies, the automated process 100 can improve client satisfaction and thereby improve business reputation.

Referring back to FIG. 1, after generating figures and/or tables at step 108, the automated process 100 can move to step 110 and identify relevant discussion boxes. A discussion box is a pre-written text box. In one or more examples, the discussion boxes can be written to generically discuss common analyses performed. For example, if a given type of feasibility report commonly includes discussion of tablet weight, discussion of table weight can be standardized such the discussion included in the report will always follow the same format and discuss the table weight results in the same manner.

In one or more examples, the discussion boxes can include one or more modifiable handles. The modifiable handles can act as replaceable blanks defining specific locations within a given discussion box that a certain data value should be discussed. Accordingly, the modifiable handles can act as variables to be populated with appropriate data values. For example, a discussion box can include the following:

Table ______ shows the tablet weights for ______. It is observed that the tablet weight was ______ across the sampling times with an average value of ______ (target: ______).

Thus, when populating the modifiable handles of the discussion box, the appropriate information and data can be automatically populated into the appropriate location within the text of the discussion box.

At step 110, in one or more examples, identifying relevant discussion boxes can include assessing what type of data was collected and which analyses have been performed and selecting corresponding discussion boxes that are associated with that data and/or analyses.

In one or more examples, the automated process 100 can generate various types of reports based on the type of analysis performed, as discussed above. Discussion boxes can be written to provide appropriate discussion for each type of report. In one or more examples, certain discussion boxes will only be relevant to certain types of reports. Thus, in one or more examples, not all discussion boxes that have been pre-written will be identified as relevant at step 110.

In one or more examples, the discussion boxes can be written in advance by one or more highly skilled individuals, with the automated process 100 configured to include each relevant discussion box each time it generates a report. Accordingly, by including discussion written by highly skilled individuals, the automated process 100 can improve the quality of the reports generated. Moreover, by standardizing the discussion included in each report and automatically including relevant discussion in each report, the automated process 100 removes the inconsistencies inherent in the prior individual-driven manual process. By removing these inconsistencies and generating high quality reports, the automated process 100 can improve client satisfaction and thereby improve business reputation.

After identifying relevant discussion boxes at step 110, the automated process 100 can move to step 112 and compile a batch report (e.g., a feasibility report). In one or more examples, to compile the batch report at step 112, the automated process 100 can develop one or more typesetting instructions. The typesetting instructions can comprise specific instructions regarding populating the relevant discussion boxes identified at step 110, embedding the figures and/or tables generated at step 108 at appropriate locations based on the relevant discussion boxes, and generating the batch report. In one or more examples, the typesetting instructions can be compiled using a typesetting document preparation system. The document preparation system can be configured to standardize the formatting of the batch report according to scientific standards and formats, which can improve the report quality. In one or more examples, the typesetting instructions can be saved in a memory in a location and/or format that can be accessed by scientists and/or users. The scientists and/or users can thus review the typesetting instructions that were used to generate the final batch report and make any necessary or desirable edits to the instructions, which can be used to generate a new version of the batch report. For example, the typesetting instructions can be stored in the format of LaTeX™ codes, a Microsoft® Word® document or Excel® spreadsheet, etc.

After compiling the batch report at step 112, the automated process 100 can move to step 114 and display the batch report. The batch report can be displayed on a user device and/or any suitable computing device. The involvement of any individuals in the automated process 100 can be restricted to executing the automated process and reviewing the displayed batch report. Accordingly, the automated process 100 can greatly reduce the effort required by each individual to generate high quality reports. Moreover, while a report is being generated, employees can focus their attention elsewhere and thus increase productivity.

The batch report displayed at step 114 of automated process 100 can include a variety of sections. Such sections can include, for example, an executive summary section, a section discussing process flow, equipment, and master formulations, a section discussing manufacturing steps and results, a section discussing analytical results, a section discussing conclusions, and a section containing references for the report. In one or more examples, the sections included in a batch report can be presented in a table of contents.

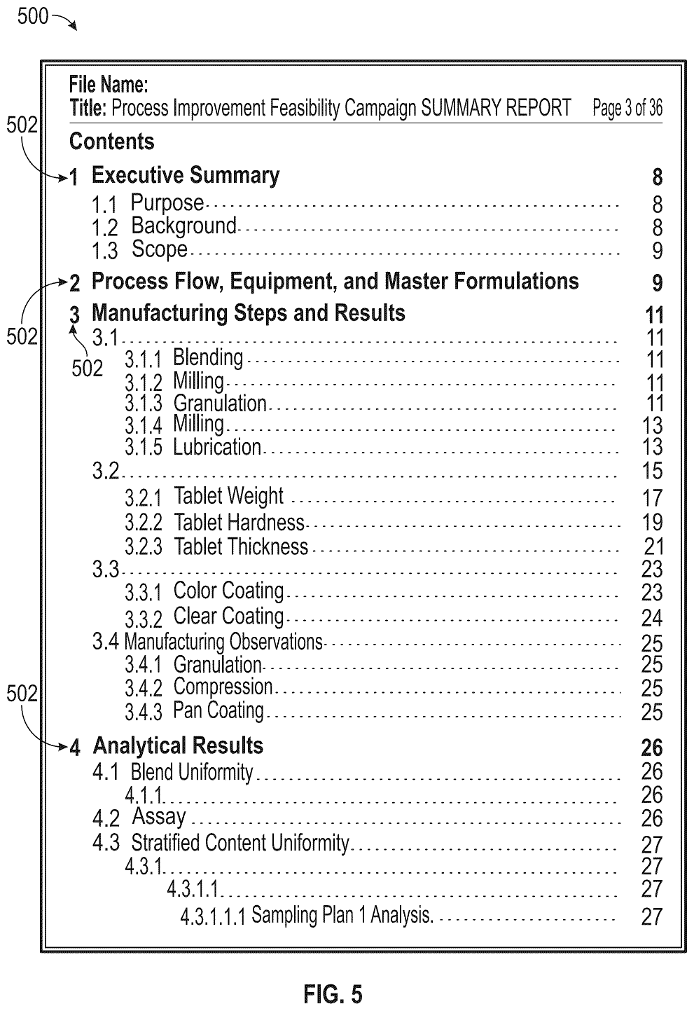

FIG. 5 depicts an exemplary user interface comprising an exemplary table of contents page 500 generated using an automated data analysis method, in accordance with some examples. Table of contents page 500 can be part of a report generated and displayed via automated process 100. As shown in FIG. 5, the table of contents page 500 includes a list of the sections 502 of the batch report. The list of sections of a batch report can include page numbers of each section as well as any other relevant details. In one or more examples, a table of contents page 500 can precede a listing of figures and/or a listing of tables contained within a report. The listing of figures and listing of tables can include a figure/table number, a figure/table name, and a figure/table page number where the corresponding figure/table can be found within the report.

An executive summary section of a report displayed at step 114 of automated process 100 can include a discussion of the purpose of a given report, relevant background information, and information regarding the scope of the report. For example, the purpose of a report can include documenting the results of a process development study of a particular pharmaceutical composition. Alternatively, the purpose of a report can include summarizing observations during the manufacturing of a particular pharmaceutical composition. Relevant background information can, in non-limiting examples, include information regarding the pharmaceutical that is the focus of the report and/or discussion or summary of the manufacturing history of the pharmaceutical. Information regarding the scope of a given report can include specific information regarding the number of batches and/or batch size (number of tablets) of a particular pharmaceutical. The scope section can include, for example, a table which contains overview information for one or more manufacturing processes such as granulation, compression, pan coating, etc.

A section of a report displayed at step 114 of automated process 100 that discusses process flow, equipment, and master formulations can include a figure illustrating the process flow of a given manufacturing process. For example, the figure illustrating the process flow can depict a timeline view of the various manufacturing steps, including the duration of each step, constituent ingredients and composition percentages, manufacturing process, etc. The process flow, equipment, and master formulations section can also include one or more tables that contain information relating to the number and types of equipment used, pharmaceutical formulation compositions for each type of pharmaceutical (i.e. for granulated tablets, compressed tablets, and/or pan-coated tablets). The process flow, equipment, and master formulations section can include discussion, tables, and/or figures based on data extracted at step 102 of automated process 100, generated at step 108 of process 100, and compiled at step 112 of process 100.

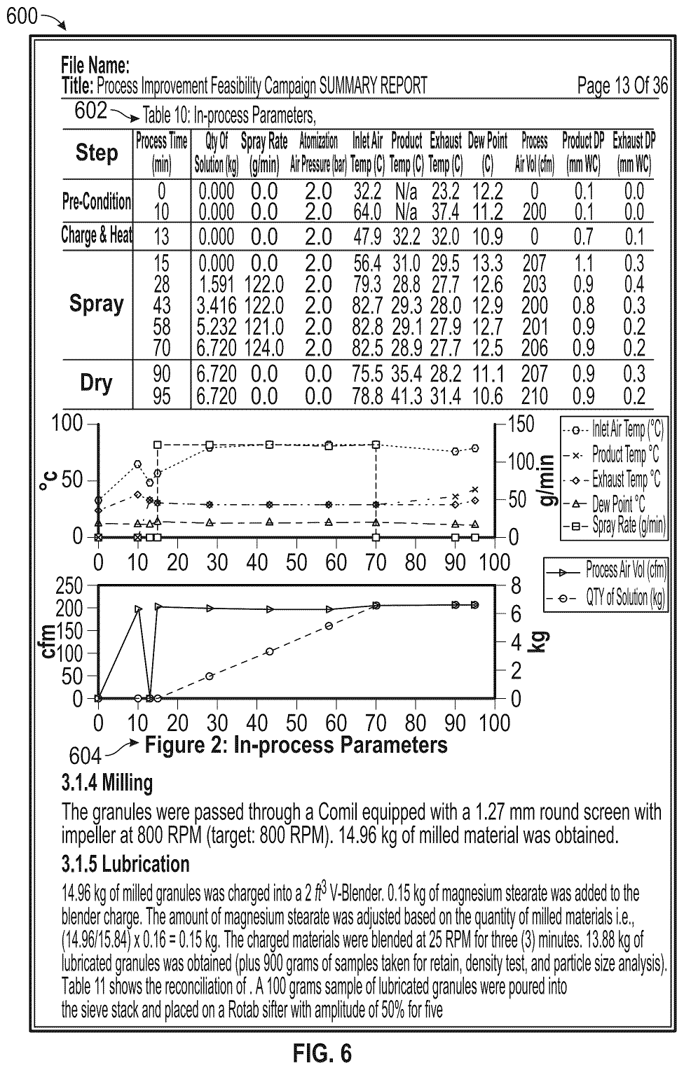

A section of a report displayed at step 114 of automated process 100 that discusses manufacturing steps and results can include discussion of blending, milling, granulation, and/or lubrication, etc. For example, FIG. 6 depicts an exemplary user interface comprising exemplary page 600 from a manufacturing steps and results section of a report generated using an automated data analysis method, in accordance with some examples. Page 600 can be part of a manufacturing steps and results section of a report generated and displayed via automated process 100.

As shown in FIG. 6, the page 600 includes a table 602, which shows various in-process parameters for a given pharmaceutical batch including the quantity of solution, spray rate, atomization air pressure, inlet air temperature product temperature, etc. In one or more examples, the in-process parameters can be graphically depicted in one or more figures. For example, the FIG. 604 of page 600 shows various parameters graphically depicted over time. Other figures which may be included in the manufacturing steps and results section can include, in non-limiting examples, figures related to particle size, in-process compression forces, tablet weight statistics, tablet hardness statistics, tablet thickness statistics, etc. The manufacturing steps and results section can include discussion, tables, and/or figures based on data extracted at step 102 of automated process 100, generated at step 108 of process 100, and compiled at step 112 of process 100.

A section of a report displayed at step 114 of automated process 100 that discusses analytical results can include sub-sections relating to blend uniformity, assay, stratified content uniformity, dissolution, water content, disintegration time, enhanced sampling, etc. In each subsection, the report can include discussion, tables, and/or figures based on data extracted at step 102 of automated process 100, generated at step 108 of process 100, and compiled at step 112 of process 100.

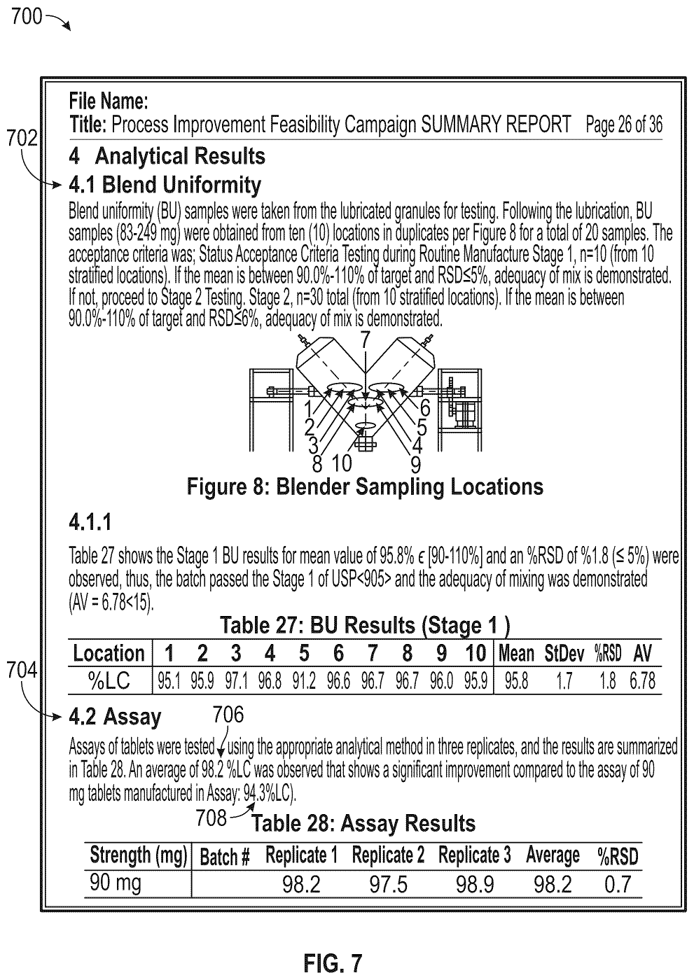

FIG. 7 depicts an exemplary user interface comprising an exemplary page 700 from an analytical results section of a report generated using an automated data analysis method, in accordance with some examples. Page 700 can be part of an analytical results section of a report generated and displayed via automated process 100. As shown in FIG. 7, the page 700 includes a sub-section regarding blend uniformity 702 and a sub-section regarding assays 704. Each sub-section of the analytical results section can include discussion, tables, and/or figures based on data extracted at step 102 of automated process 100, generated at step 108 of process 100, and compiled at step 112 of process 100. In one or more examples, the sub-sections can also include analytical conclusions that have been automatically generated via automated process 100. For example, as shown in subsection 704, this report page 700 provides a calculated average assay of a first percentage 706 which is characterized as “a significant improvement compared” to an average value of a second percentage 708 from a different manufacturing batch. Thus, the report displayed at step 114 of automated process 100 not only includes the results of statistical analyses that were performed automatically, but also automatically provides meaningful discussion of those results.

A section of a report displayed at step 114 of automated process 100 that discusses conclusions can, in one or more examples, include discussion of one or more recommendations that are generated based on conclusions of the report. For example, the report may determine that the data analysis revealed a low assay issue and the report may contain one or more recommendations to alter the granulation process in order to minimize the possibility of a recurrence of the low assay issue.

A section of a report displayed at step 114 of automated process 100 that contains references for a given report can include references to one or more documents used to formulate the report. Such documents can include, for example, specific batch reports identified by a document number, regulatory and/or company protocols and/or standards identified by a document number.

As discussed above, the raw data necessary to generate a given batch report can be contained within batch production records that are either in a printed document or PDF document format due to regulatory necessity. Whereas previously this raw data was extracted by two individuals with one copying values into a spreadsheet and one verifying the accuracy of that process, the automated process 100 can perform such extraction entirely independently. In one or more examples, the raw data necessary to generate a given batch report can be contained within batch production records exported from a machine in a comma-separated value (CSV) format. The automated process 100 can be configured to extract such values directly from a CSV file.

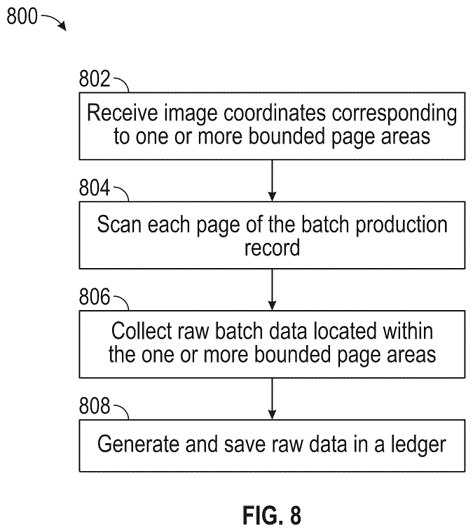

FIG. 8 depicts an exemplary automated process 800 to extract raw batch data from a printed or PDF batch production report, in accordance with one or more examples. Automated process 800 can be performed as part of step 102 of automated process 100. Automated process 800 can begin with step 802, by receiving image coordinates corresponding to one or more bounded page areas. In one or more examples, a batch production record exported from a certain type of machine will be exported with relevant raw data values located within a certain area of a page. Accordingly, the image coordinates can correspond to the area within the page wherein raw data values are recorded. In one or more examples, a user may be able to edit the image coordinates corresponding to any changes to the certain area where relevant raw data values are located within a batch production record. In one or more examples, the user may be able to perform such updates in real-time as batch production records are received.

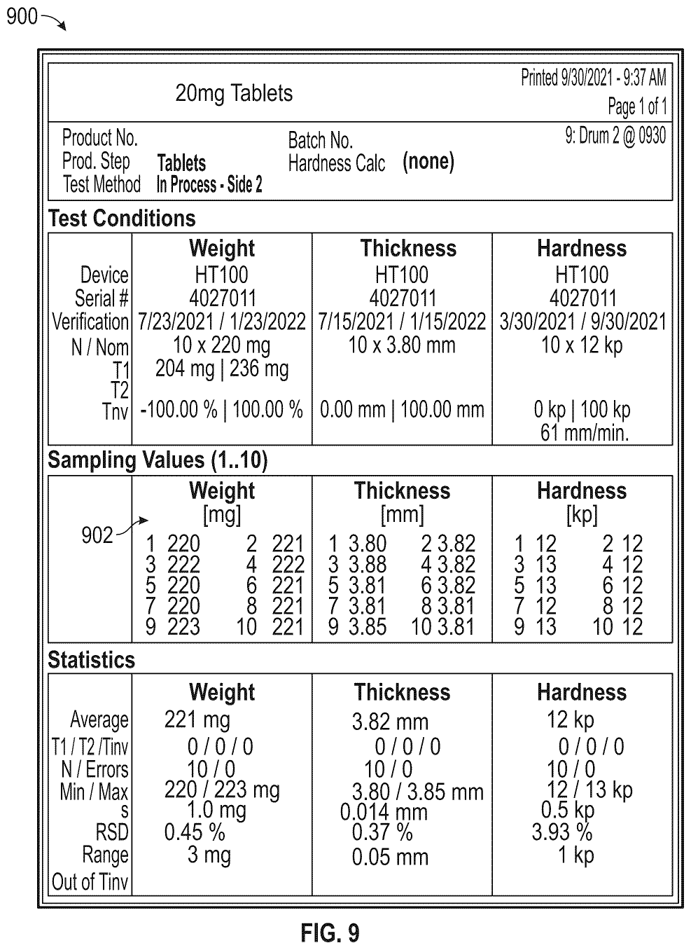

FIG. 9 depicts an exemplary batch production report 900, in accordance with one or more examples. As shown in FIG. 9, the relevant raw data pertaining to sampling values for weight, thickness, and hardness, are contained within a certain region 902 of the batch production report 900. Accordingly, the image coordinates received at step 802 of automated process 800 can include image coordinates corresponding to that region 902.

Referring back to FIG. 8, after receiving image coordinates at step 802, the automated process 800 can move to step 804 and scan each page of the batch production record. Scanning of the batch production record can be performed any conventional scanning technology known in the art.

After scanning each page of the batch production record at step 804, the automated process 800 can move to step 806 and collect the raw batch data located within the one or more bounded page areas. The one or more bounded page areas can correspond to the image coordinates received at step 802.

After collecting the raw batch data at step 806, the automated process 800 can move to step 808 and generate a ledger that contains the raw data. The ledger can be saved in a location and/or format that is accessible to scientists and/or users. For example, the raw data may be saved as a spreadsheet within a commercial spreadsheet software program such as Microsoft® Excel®.

In one or more examples, automated process 800 and automated process 100 can be performed in real-time as data production records are output from one or more manufacturing devices. Step 106 of automated process 100, of performing statistical analyses on stored raw batch data, may thus be performed in real-time as a given batch is being manufactured. As part of performing statistical analyses at step 106, automated process 100 can include identifying any outlier data values within the data. Identifying one or more outlier data values can, in one or more examples, involve conducting a variance component analysis.

Upon identifying an outlier data value, the automated process 100 can be configured to provide an alert to a user. In one or more examples, the automated process 100 can be configured to determine whether the outlier value is caused by a manufacturing process error or a post-manufacturing data analysis error. In one or more examples, the automated process 100 can be configured to provide one or more guidelines to adjust a setting of one or more manufacturing devices. For example, the automated process 100 may be configured to provide a guideline to stop a manufacturing device upon identifying an outlier value that is attributable to a manufacturing process error.

Previously if data analyses regarding a given production batch contained an outlier data value, the entire batch may have been disposed of. Accordingly, by determining whether an outlier value is attributable to a data analysis error, the automated process 100 can ensure that any batches with such values are not disposed of. Moreover, by identifying outlier values in real-time, the automated process 100 can enable users to address any manufacturing process errors immediately. Previously, an outlier value that was attributable to a manufacturing error may have been identified after the entire batch was produced, thus leading to the disposal of that batch. By enabling users to address manufacturing errors in real-time and/or by providing a guideline to adjust one or more settings of manufacturing devices, however, the automated process 100 can halt a given process and ensure that additional resources are not used to produce the batch until the error is addressed.

As discussed above, whereas previously manually drafting a report was a time-intensive and analytically difficult process for a user, using an automated process such as process 100 can enable a user to generate a better and more consistent report in a much shorter period. The user interaction in the automated process can be reduced to simply inputting some data, commanding the automated process to run, and reviewing the output.

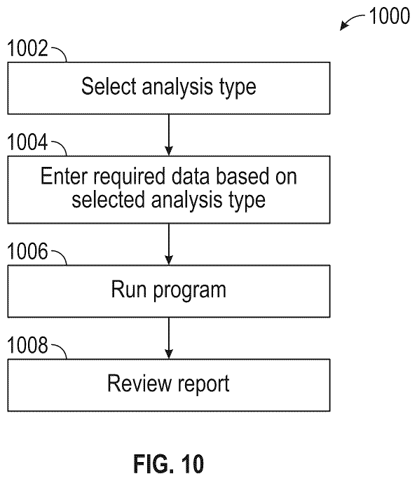

FIG. 10 depicts an exemplary process 1000 for a user to command the automated generation of a batch report, in accordance with one or more examples. Process 1000 can be used to command the automated generation of a batch report using automated process 100. Process 1000 can be implemented using one or more suitable computing devices capable of displaying a user interface to a user and recording and/or transmitting user inputs to the user interface. The computing device that enables a user to perform method 1000 can be the same computing device that performs the automated process 100, or it can be communicatively coupled to the computing device that is performing the automated process 100.

In one or more examples, the process 1000 can begin at step 1002 with a user selecting an analysis type. A user can use a user interface, such as user interface 200 of FIG. 2 to select an analysis type. After selecting the analysis type at step 1002, the process 1000 can move to step 1004 wherein the user enters required data based on the selected analysis type. The user interface for a user to enter required data can be user interface 300 of FIG. 3. As discussed above, the user interface that the user uses to enter required data based on the selected analysis type at step 1004 can allow the user to input, for example, a directory location, a product name, one or more data fields based on the selected analysis type, etc.

After entering the required data based on the selected analysis type at step 1004, the process 1000 can move to step 1006 wherein the user runs the program. Upon selecting to run the program at step 1006, a user command may be generated that triggers an automated process such as process 100 to generate a batch report. In one or more examples, upon selecting to run the program at step 1006, a user command may be generated and received at step 104 of process 100 of FIG. 1. After running the program at step 1006, the process 1000 can move to step 1008 wherein the user reviews the report generated. As discussed above, the report generated by the automated process 100 contains more sophisticated analysis and more standardized analysis and presentation of the analysis, and can be generated in a fraction of the time that a manually drafted report would have been.

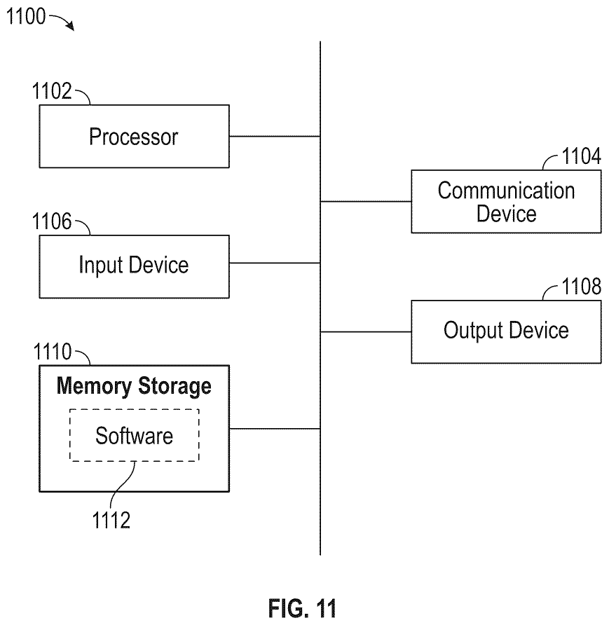

FIG. 11 illustrates an example of a computing device 1100 in accordance with one examples. Device 1100 can be a host computer connected to a network. Device 1100 can be a client computer or a server. As shown in FIG. 11, device 1100 can be any suitable type of microprocessor-based device, such as a personal computer, workstation, server, or handheld computing device (portable electronic device) such as a phone or tablet. The device can include, for example, one or more of processors 1102, input device 1106, output device 1108, storage 1110, and communication device 1104. Input device 1106 and output device 1108 can generally correspond to those described above and can either be connectable or integrated with the computer.

Input device 1106 can be any suitable device that provides input, such as a touch screen, keyboard or keypad, mouse, or voice-recognition device. Output device 1108 can be any suitable device that provides output, such as a touch screen, haptics device, or speaker.

Storage 1110 can be any suitable device that provides storage, such as an electrical, magnetic, or optical memory, including a RAM, cache, hard drive, or removable storage disk. Communication device 1104 can include any suitable device capable of transmitting and receiving signals over a network, such as a network interface chip or device. The components of the computer can be connected in any suitable manner, such as via a physical bus or wirelessly.

Software 1112, which can be stored in storage 1110 and executed by processor 1102, can include, for example, the programming that embodies the functionality of the present disclosure (e.g., as embodied in the devices as described above).

Software 1112 can also be stored and/or transported within any non-transitory computer-readable storage medium for use by or in connection with an instruction execution system, apparatus, or device, such as those described above, that can fetch instructions associated with the software from the instruction execution system, apparatus, or device and execute the instructions. In the context of this disclosure, a computer-readable storage medium can be any medium, such as storage 1110, that can contain or store programming for use by or in connection with an instruction execution system, apparatus, or device.

Software 1112 can also be propagated within any transport medium for use by or in connection with an instruction execution system, apparatus, or device, such as those described above, that can fetch instructions associated with the software from the instruction execution system, apparatus, or device and execute the instructions. In the context of this disclosure, a transport medium can be any medium that can communicate, propagate, or transport programming for use by or in connection with an instruction execution system, apparatus, or device. The transport readable medium can include, but is not limited to, an electronic, magnetic, optical, electromagnetic, or infrared wired or wireless propagation medium.

Device 1100 may be connected to a network, which can be any suitable type of interconnected communication system. The network can implement any suitable communications protocol and can be secured by any suitable security protocol. The network can comprise network links of any suitable arrangement that can implement the transmission and reception of network signals, such as wireless network connections, T1 or T3 lines, cable networks, DSL, or telephone lines.

Device 1100 can implement any operating system suitable for operating on the network. Software 1112 can be written in any suitable programming language, such as C, C++, Java, or Python. In various embodiments, application software embodying the functionality of the present disclosure can be deployed in different configurations, such as in a client/server arrangement or through a Web browser as a Web-based application or Web service, for example.

Although the disclosure and examples have been fully described with reference to the accompanying figures, it is to be noted that various changes and modifications will become apparent to those skilled in the art. Such changes and modifications are to be understood as being included within the scope of the disclosure and examples as defined by the claims.

For the purpose of clarity and a concise description, features are described herein as part of the same or separate embodiments; however, it will be appreciated that the scope of the disclosure includes embodiments having combinations of all or some of the features described.

Unless defined otherwise, all terms of art, notations and other technical and scientific terms or terminology used herein are intended to have the same meaning as is commonly understood by one of ordinary skill in the art to which the claimed subject matter pertains. In some cases, terms with commonly understood meanings are defined herein for clarity and/or for ready reference, and the inclusion of such definitions herein should not necessarily be construed to represent a substantial difference over what is generally understood in the art.

The above description is presented to enable a person skilled in the art to make and use the disclosure, and is provided in the context of a particular application and its requirements. Various modifications to the preferred embodiments will be readily apparent to those skilled in the art, and the generic principles defined herein may be applied to other embodiments and applications without departing from the spirit and scope of the disclosure. Thus, this disclosure is not intended to be limited to the embodiments shown, but is to be accorded the widest scope consistent with the principles and features disclosed herein.

1. A computer-implemented method for generating a batch report comprising:
extracting raw batch data from one or more batch manufacturing reports received from one or more manufacturing devices and storing the extracted raw batch data in a memory;
receiving a user command on a user device to automatically generate a batch report, and in response to receiving the user command:
performing one or more statistical analyses on the stored raw batch data;
generating one or more figures and one or more tables using the analyzed data and storing the one or more figures and the one or more tables in the memory;
identifying one or more discussion boxes that are relevant to the analyzed data;
compiling a batch report comprising the one or more identified relevant discussion boxes, the one or more figures, and the one or more tables;
saving the compiled batch report in the memory; and
displaying the compiled batch report on the user device.

2. The computer-implemented method of claim 1, comprising:
identifying one or more outlier values in the analyzed data; and
determining whether the one or more outlier values is caused by a manufacturing process error or a post-manufacturing data analysis error.

3. The computer-implemented method of claim 2, wherein the raw batch data is extracted from the one or more batch manufacturing reports in real-time as the one or more batch manufacturing reports are received.

4. The computer-implemented method of claim 3, comprising providing one or more guidelines to adjust one or more settings of the one or more manufacturing devices if the outlier value is caused by a manufacturing process error.

5. The computer-implemented method of claim 1, wherein the one or more batch manufacturing reports comprise a plurality of batch pages, and extracting the raw batch data comprises:
receiving one or more image coordinates corresponding to one or more bounded areas within a page;
photoscanning each page of the plurality of batch pages;
collecting raw batch data located within the one or more bounded areas on each page of the plurality of photoscanned batch pages; and
generating a ledger containing the collected raw batch data.

6. The computer-implemented method of claim 1, wherein compiling the batch report comprises generating one or more instruction codes that comprise instructions to:
populate one or more modifiable handles in the one or more relevant discussion boxes with one or more statistical data values from the analyzed data;
embed the one or more figures and the one or more tables in one or more appropriate locations based on the relevant discussion boxes; and
generate a data report comprising the one or more populated relevant discussion boxes, the one or more embedded figures, and the one or more embedded tables.

7. The computer-implemented method of claim 6, wherein the one or more instruction codes comprise LaTeX codes.

8. The computer-implemented method of claim 6, wherein the one or more modifiable handles each correspond to a data metric, the one or more discussion boxes comprise pre-written text regarding one or more data metrics, and populating the one or more modifiable handles comprises replacing each of the modifiable handles with a corresponding data metric from the analyzed data.

9. The computer-implemented method of claim 1, wherein the compiled batch report indicates whether the analyzed data is within one or more defined batch specifications.

10. A system for generating a batch report, the system comprising:
a memory;
one or more processors; and
one or more programs, wherein the one or more programs are stored in the memory and configured to be executed by the one or more processors, and the one or more programs when executed by the one or more processors cause the processor to:
extract raw batch data from one or more batch manufacturing reports received from one or more manufacturing devices and storing the extracted raw batch data in a memory;
receive a user command on a user device to automatically generate a batch report, and in response to receiving the user command:
perform one or more statistical analyses on the stored raw batch data;
generate one or more figures and one or more tables using the analyzed data and storing the one or more figures and the one or more tables in the memory;
identify one or more discussion boxes that are relevant to the analyzed data;
compile a batch report comprising the one or more relevant discussion boxes, the one or more figures, and the one or more tables;
save the compiled batch report in the memory; and
display the compiled batch report on the user device.

11. The system of claim 10, wherein the one or more programs when executed by the one or more processors cause the processor to:
identify one or more outlier values in the analyzed data; and
determine whether the one or more outlier values is caused by a manufacturing error or a post-manufacturing data analysis error.

12. The system of claim 11, wherein the raw batch data is extracted from the one or more batch manufacturing reports in real-time as the one or more batch manufacturing reports are received.

13. The system of claim 12, wherein the one or more programs when executed by the one or more processors cause the processor to provide one or more guidelines to adjust one or more settings of the one or more manufacturing devices if the outlier value is caused by a manufacturing process error.

14. The system of claim 10, wherein the one or more batch manufacturing reports comprise a plurality of batch pages, and extracting the raw batch data comprises:
receiving one or more image coordinates corresponding to one or more bounded areas within a page;
photoscanning each page of the plurality of batch pages;
collecting raw batch data located within the one or more bounded areas on each page of the plurality of photoscanned batch pages; and
generating a ledger containing the collected raw batch data.

15. The system of claim 10, wherein compiling the batch report comprises generating one or more instruction codes that comprise instructions to:
populate one or more modifiable handles in the one or more relevant discussion boxes with one or more statistical data values from the analyzed data;
embed the one or more figures and the one or more tables in one or more appropriate locations based on the relevant discussion boxes; and
generate a data report comprising the one or more populated relevant discussion boxes, the one or more embedded figures, and the one or more embedded tables.

16. The system of claim 15, wherein the one or more instruction codes comprise LaTeX codes.

17. The system of claim 15, wherein the one or more modifiable handles each correspond to a data metric, the one or more discussion boxes comprise pre-written text regarding one or more data metrics, and populating the one or more modifiable handles comprises replacing each of the modifiable handles with a corresponding data metric from the analyzed data.

18. The system of claim 10, wherein the compiled batch report indicates whether the analyzed data is within one or more defined batch specifications.

19. A computer-readable storage medium storing one or more programs for generating a batch report, the one or more programs comprising instructions which, when executed by an electronic device with a display and a user input interface, cause the device to:
extract raw batch data from one or more batch manufacturing reports received from one or more manufacturing devices and storing the extracted raw batch data in a memory;
receive a user command on a user device to automatically generate a batch report, and in response to receiving the user command:
perform one or more statistical analyses on the stored raw batch data;
generate one or more figures and one or more tables using the analyzed data and storing the one or more figures and the one or more tables in the memory;
identify one or more discussion boxes that are relevant to the analyzed data;
compile a batch report comprising the one or more relevant discussion boxes, the one or more figures, and the one or more tables;
save the compiled batch report in the memory; and
display the compiled batch report on the user device.

20. The computer-readable storage medium of claim 19, wherein the one or more programs when executed by the electronic device cause the device to:
identify one or more outlier values in the analyzed data; and
determine whether the one or more outlier values is caused by a manufacturing error or a post-manufacturing data analysis error.

21. The computer-readable storage medium of claim 20, wherein the raw batch data is extracted from the one or more batch manufacturing reports in real-time as the one or more batch manufacturing reports are received.

22. The computer-readable storage medium of claim 21, wherein the one or more programs when executed by the electronic device cause the device to provide one or more guidelines to adjust one or more settings of the one or more manufacturing devices if the outlier value is caused by a manufacturing process error.

23. The computer-readable storage medium of claim 19, wherein the one or more batch manufacturing reports comprise a plurality of batch pages, and extracting the raw batch data comprises:
receiving one or more image coordinates corresponding to one or more bounded areas within a page;
photoscanning each page of the plurality of batch pages;
collecting raw batch data located within the one or more bounded areas on each page of the plurality of photoscanned batch pages; and
generating a ledger containing the collected raw batch data.

24. The computer-readable storage medium of claim 19, wherein compiling the batch report comprises generating one or more instruction codes that comprise instructions to:
populate one or more modifiable handles in the one or more relevant discussion boxes with one or more statistical data values from the analyzed data;
embed the one or more figures and the one or more tables in one or more appropriate locations based on the relevant discussion boxes; and
generate a data report comprising the one or more populated relevant discussion boxes, the one or more embedded figures, and the one or more embedded tables.

25. The computer-readable storage medium of claim 24, wherein the one or more instruction codes comprise LaTeX codes.

26. The computer-readable storage medium of claim 24, wherein the one or more modifiable handles each correspond to a data metric, the one or more discussion boxes comprise pre-written text regarding one or more data metrics, and populating the one or more modifiable handles comprises replacing each of the modifiable handles with a corresponding data metric from the analyzed data.

27. The computer-readable storage medium of claim 19, wherein the compiled batch report indicates whether the analyzed data is within one or more defined batch specifications.
